SEQUENCE LISTING (1 sequence)

SEQ ID NO: 1 - DNA, 10 bp, artificial
  CLEAR Consensus Sequence
gtcacgtgac

(19) United States
(12) Patent Application Publication
(10) Pub. No.: US 2020/0385756 A1
(43) Pub. Date: Dec. 10, 2020

(54) OPTOGENETIC GENE EXPRESSION SYSTEMS AND METHODS

(71) Applicant: STC.UNM, Albuquerque, NM (US)

(72) Inventors: Kiran Bhaskar, Albuquerque, NM (US); Jessica Lee Binder, Albuquerque, NM (US); Jason Weick, Albuquerque, NM (US)

(21) Appl. No.: 16/893,964

(22) Filed: Jun. 5, 2020

Related U.S. Application Data
(60) Provisional application No. 62/858,533, filed on Jun. 7, 2019.

(51) Int. Cl. C12N 15/85; C07K 14/47; C12N 13/00

(52) U.S. Cl. CPC C12N 15/85; C12N 2800/107; C12N 13/00; C07K 14/4705

(57) ABSTRACT

A genetic expression system generally includes a polynucleotide that encodes Transcription Factor EB (TFEB) under transcriptional control of a promoter, and a polynucleotide that encodes a light-activatable protein that binds to the promoter in the presence of light but does not bind to the promoter in the presence of light.

### CROSS-REFERENCE TO RELATED APPLICATION

This application claims the benefit of U.S. Provisional Patent Application No. 62/858,533, filed Jun. 7, 2019, which is incorporated herein by reference in its entirety.

### GOVERNMENT FUNDING

This invention was made with government support under NS093442 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “310148US01 ST25.txt” having a size of 422 bytes and created on Jun. 4, 2020. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a genetic expression system. Generally, the genetic expression system includes a polynucleotide that encodes Transcription Factor EB (TFEB) under transcriptional control of a promoter, and a polynucleotide that encodes a light-activatable protein that binds to the promoter in the presence of light but does not bind to the promoter in the absence of light.

In some embodiments, the promoter is the cytomegalovirus (CMV) promoter.

In some embodiments, the TFEB includes, at its C-terminus, the cMyc nuclear localization signal (NLS)

In some embodiments, the light-activatable protein includes a complex that includes Light-Oxygen-Voltage (LOV) protein and a dimerizable Helix-Turn-Helix (HTH) DNA-binding domain.

In another aspect, this disclosure describes a cell that includes any embodiment of the genetic expression system summarized above. In some embodiments, the call may be a neuron. In some of these embodiments, the neuron may be a cell at risk of displaying a tauopathy.

In another embodiment, this disclosure describes a method of treating a neuron at risk of displaying a tauopathy. Generally, the method includes introducing into the cell an embodiment of the genetic expression system summarized above, and exposing the cell to light effective to cause the light-activatable protein to bind to the promoter, thereby expressing TFEB.

In some embodiments, exposing the cell to light can involve exposing the cell to light for a period sufficient for the genetic expression system to produce a protein that promotes autophagy of the neuron. In some of these embodiments, promoting autophagy of the neuron can involve reducing pathological tau protein in the neuron.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Optogenetic gene expression system in neuronal cell line. (A) Schematic of previously established gene expression system derived from an EL222 bacterial transcription factor, termed Light-Activated Protein (LAP). (B) Schematic of changes made to the LAP construct for successful neuronal transfection/induction as well as TFEB cloned into the LRE construct.

FIG. 2. Optogenetic gene expression system in neuronal cell line. (A) Images quantifying expression of pLRE-Firefire luciferase reporter (pLRE-FLuc) in HEK293 cells. (B) Bar graph measuring quantification of relative luciferase units (RLU) radiance levels detected by IVIS (mean+s.e.m, unpaired Student's t test or one-way ANOVA with Tukey multiple comparison test, ****p<0.0005 n=5) in HEK293 cells. (C) Images quantifying expression of pLRE-Firefire luciferase reporter (pLRE-FLuc) in N2a cells. (D) Bar graph measuring quantification of relative luciferase units (RLU) radiance levels detected by IVIS (mean+s.e.m, unpaired Student's t test or one-way ANOVA with Tukey multiple comparison test, ****p<0.0005 n=5) in N2a cells.

FIG. 3. TFEB differentially targets various forms of pTau. (A) Western blot showing reduction in various forms of tau via WT-0N3R, (0N3R) T231D/S235D, (0N4R) P301L, and WT-0N4R with constitutive overexpression of TFEB activity. (B) Quantification of the reduction of various forms of tau via WT-0N3R, (0N3R) T231D/S235D, (0N4R) P301L, and WT-0N4R with the addition of constitutive overexpression of TFEB activity. Results indicated most forms of tau are equivalently reduced by TFEB, however (0N3R) T231D/S235D shows highest significance in expression and reduction. Total tau/GAPDH ratio (mean+s.e.m, Student's t test, **p<0.01 n=3). (C) Western blot showing reduced (0N3R) T231D/S235D with various forms of constitutive TFEB overexpression: pCMV-TFEB3×FLAG, pCMV-TFEB-GFP, pCMV-TFEB(S211A)GFP. (D) Quantification of the reduced (0N3R) T231D/S235D with the various forms of constitutive TFEB overexpression. Results indicate pCMV-TFEB(S211A)GFP holds the best yield in total tau reduciton. Total tau/GAPDH ratio (mean+s.e.m, one-way ANOVA with Tukey multiple comparison test, ***p<0.005 n=4) E-F. With the addition of Bafilomycin, Western blot and quantification showing an increased trend in LC3-II levels in pCMV-TFEB(S211A)GFP. LC3-II/GAPDH ratio (mean+s.e.m, one-way ANOVA with Tukey multiple comparison test, n=3).

FIG. 4. Optogenetic TFEB induction in neuronal cell line and CLEAR activity readout. Immunocytochemistry images showing significant increase in TFEB expression in Light control versus Dark. Scale bar: 20 μm.

FIG. 5. Optogenetic TFEB induction in neuronal cell line and CLEAR activity readout. (A) Comparison of various versions of LAP constructs using pLRE-TFEB-(S211A)GFP (mean+s.e.m, Student's t test, ****p<0.0005, n=4). (B) Images comparing various versions of LAP constructs using pCLEAR-Firefly Luciferase reporter, (pCLEAR-Fluc) in N2a cells. (C) Quantitative comparison of various versions of LAP constructs using pCLEAR-Firefly Luciferase reporter, (pCLEAR-Fluc) in N2a cells measuring luciferase activity units (RLU) via radiance levels detected by IVIS (mean+s.e.m, Student's t test or one-way ANOVA with Tukey multiple comparison test, ****p<0.0005 n=4).

FIG. 6. Optogenetic TFEB induction in neuronal cell line reduces neuronal pathological mimicking tau. (A) Western Blot analysis showing overall total protein levels are reduced when Opto-TFEB is expressed via light stimulation compared to dark. (B) The TFEB-GFP/GAPDH ratio was significantly elevated (**p<0.001; ****p<0.0001; unpaired t test, n=3) in N2a cells expressing T231D/S235D phosphorylation-mimicking tau along with either constitutively expressing pCMV-TFEB (S211A)GFP or blue-light induced TFEB(S211A) GFP (pCMV-LAP-2×NLS+pLRE-TFEB(S211A)GFP+Light) compared to their respective control groups. (C) The Tau12/GAPDH ratio was significantly reduced (*=p<0.001; ****p<0.0001; unpaired t test, n=4) in N2a cells expressing T231D/S235D phosphorylation-mimicking tau along with either constitutively expressing pCMV-TFEB (S211A)GFP or blue-light induced TFEB(S211A) GFP (pCMV-LAP-2×NLS+pLRE-TFEB(S211A)GFP+Light) compared to their respective control groups.

FIG. 7. Optogenetic TFEB induction in neuronal cell line reduces neuronal pathological mimicking tau. (A) CELLOMICS (Thermo Fisher Scientific, Inc., Waltham, Mass.)-based high-content imaging analysis of the effects on total Tau levels within Dark and Light controls. Cells were automatically identified based on nuclear staining (DAPI), then cells were selected for positive nuclear green fluorescence (TFEB(S211A)GFP) to further analyze for Tau12 (RED) intensity levels within 100 pixel radius per cell. Briefly, white lines represent cell boundaries, red lines represent positive cytosolic Tau12, and yellow lines indicate nuclear TFEB(S211A)GFP-positive cells, then subjected by automated image analysis. (B) CELLOMICS (Thermo Fisher Scientific, Inc., Waltham, Mass.)-based high-content quantitative morphometry showing significant increase (****p<0.0001; unpaired t test, n=3) in the nuclear TFEB(S211A)GFP in N2a cells expressing T231D/S235D phosphorylation-mimicking tau along with blue-light induced TFEB(S211A) GFP (pCMV-LAP-2×NLS+pLRE-TFEB(S211A)GFP+Light) compared to ‘no light’ (dark) control group. (C) CELLOMICS (Thermo Fisher Scientific, Inc., Waltham, Mass.)-based high-content quantitative morphometry showing significant decrease (****p<0.0001; unpaired t test, n=3) in the cytosolic Tau12 (red) in N2a cells expressing T231D/S235D phosphorylation-mimicking tau along with blue-light induced TFEB(S211A) GFP (pCMV-LAP-2×NLS+pLRE-TFEB(S211A)GFP+Light) compared to ‘no light’ (dark) control group. (D) Representation of colocalization profile for Tau12 (red) and LRE-TFEB(S211A)GFP (green) analysis. Quantitative confocal immunocytochemistry using N2a cells overexpressing human 0N3R-T231D/S235D tau show lack of colocalization of optogenetically induced TFEB expression with Tau12 positive cells. Quantitative morphometric data (mean+s.e.m, Student's t test, ****p<0.0001, n=3). Scale bars: 10 μm in (A); 20 μm in D.

FIG. 8. Optogenetic TFEB clears pTau in human induced pluripotent stem cells derived into neurons (iPSNs). (A) Immunohistochemistry images showing increase in TFEB expression with subsequent lower levels of p-Tau (AT8 and AT180) within betalll-tubulin (neurons) in Light control compared to Dark, using viral-particle versions, pGF1-CMV-LAP-2×NLS and pGF1-LRE-TFEB-(S211A)GFP. Scale bars: 20 μm. (B) Quantitative immunocytochemistry showing significant increase in TFEB expression imaged in (A). Mean+s.e.m, Student's t test, *p<0.05, n=8).

FIG. 9. Optogenetic TFEB clears pTau in human induced pluripotent stem cells derived into neurons (iPSNs). Two-day timeline using RT-qPCR analysis of TFEB gene expression and TFEB targets (PTEN, CTSF, and MCOLN1). Compared to Dark, each sample was taken 24 hours of subsequent time-point. On Day-1, 12-hour light stimulation; Day-2 from same sample, light was off.

FIG. 10. Optogenetic TFEB clears pTau in human induced pluripotent stem cells derived into neurons (iPSNs). Western blot of protein expression corresponding to RT-qPCR samples in FIG. 9. Increased GFP (TFEB) levels and reduced pMAPT (AT8 and AT180) with the transduction of viral optogenetic TFEB and subsequent light stimulation. (mean+s.e.m, one-way ANOVA, ***p<0.0005, n=3-6).

FIG. 11. Optogenetic TFEB clears pTau in human induced pluripotent stem cells derived into neurons (iPSNs). (A) Quantification of protein expression corresponding to RT-qPCR samples in FIG. 9, normalized to GAPDH or action. (B) Quantification of protein expression corresponding to RT-qPCR samples in FIG. 9, normalized to Tau12. (mean+s.e.m, one-way ANOVA with Dunnett's multiple comparison test, *p<0.05; ***p<0.0005, n=3-6).

FIG. 12. Schematic diagram of in vivo optogenetic induction of autophagy in neurons.

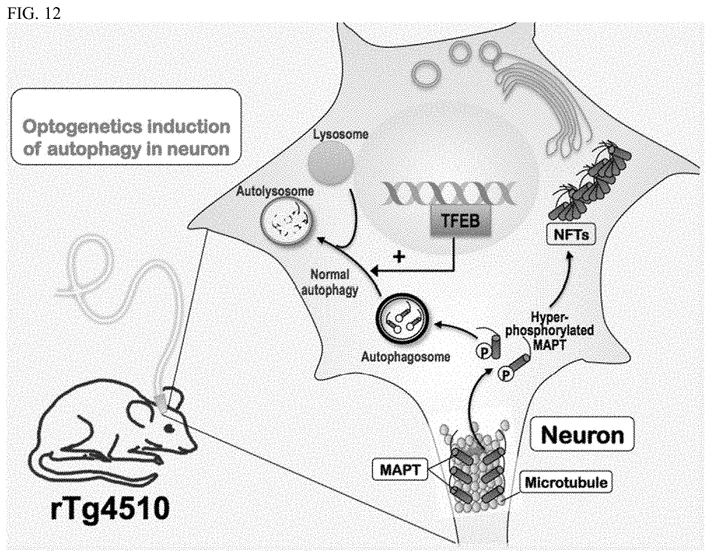

### DETAILED DESCRIPTION OF ILLUSTRATIVE EMBODIMENTS

Among the various microtubule-associated proteins (MAP), tau (encoded by MAPT) predominately localizes to axons where it binds to microtubules. Tau promotes nucleation, promotes stabilization, and inhibits disassembly of microtubules. However, tau is susceptible to many post-translational modifications, with phosphorylation being one of the well-studied modifications. Upon hyperphosphorylation, tau linearly decreases its affinity to microtubules, causing depolymerization. These dissociated forms of tau can self-assemble into paired-helical filaments (PHFs) gaining further potential to aggregate as Neurofibrillary tangles (NFTs)—a classical neuropathological hallmark of Alzheimer's disease (AD) and related tauopathies. Alternatively, hyperphosphorylated and pathological tau (p-Tau) has been shown to acquire gain-of-toxic function in triggering synaptotoxicity relevant to AD. While AD a common form of tauopathy, NFT pathology is also the primary etiology in many other tauopathies, such as Progressive Supranuclear Palsy (PSP), Pick's disease (PiD), Corticobasal Degeneration (CBD), Fronto-temporal Dementia, Parkinsonism linked to Chromosome-17 tau-type (FTDP-17T) and others. Because of the rise in tauopathy related deaths, there is an urgent need to find interventions against tauopathies.

A plausible strategy to prevent p-Tau from becoming pathological is to promote its degradation via autophagy in “at risk” neuronal populations. As used herein, the term “at risk” refers to a subject or, as the case may be, a population of neurons, that may or may not actually possess the described risk, but possess at least one indicia of the described risk compared to individuals (or neurons) that lack the one or more indicia, regardless of the whether the individual (or an individual having the “at risk” population of neurons) manifests any symptom or clinical sign associated with the described risk.

This disclosure describes a light-inducible gene expression system and methods of controlling gene expression to reduce pathological tau in an in vitro model. While described in the context of a model exemplary embodiment in which blue light (465 nm) is used to induce Transcription Factor EB (TFEB) gene expression.

In a model embodiment, the system includes a blue-light-inducible TFEB gene expression system that is exemplified in mouse neuronal cell lines and human AD iPSCs derived into mature neurons (iPSNs). The model embodiment demonstrates successful light-controlled gene expression and effectively enhances autophagy flux, specifically targeting and reducing pathological tau in human AD iPSNs.

Impairment of autophagic processes has been implicated in several neurodegenerative disorders. Autophagy's potential in clearing p-Tau may involve one or more of the following: (i) reversing a causal link between failure in autophagic processing and AD-related pathologies, (ii) autophagy disposes of potentially toxic intracellular protein aggregates too large for proteasomal removal and performs trophic functions (sometimes referred to as “programmed cell survival”), (iii) autophagy removes depolarizes mitochondria and shows cytoprotective interactions with stressed endoplasmic reticulum that are of direct consequence for tauopathies, (iv) autophagy inhibits spurious inflammasome activation, which, when left uncontrolled, could drive tau pathology and cognitive impairment, and (v) autophagic processing can enhance clearance of p-Tau and rescue neurotoxicity in a mouse model of tauopathy.

This disclosure describes inducing autophagy using a light-inducible genetic expression system. Transcription Factor EB (TFEB) regulates transcription of an entire CLEAR (Coordinated Lysosomal Expression and Regulation) network, which consists of a consensus site predominately found in the promoter regions of autophagy-lysosomal genes. Thus, when TFEB localization is nuclear, it leads to robust increase in lysosome biogenesis, and results in accelerated degradation of autophagic substrates. Phosphorylation of Ser211 in TFEB by mammalian target of rapamycin complex 1 or mechanistic target of rapamycin complex 1 (mTORC1) is one of the regulators of nuclear localization, as the pS211 phosphorylation inhibits TFEB entry into the nucleus. Strikingly, S211A mutation facilitates TFEB's nuclear entry and activation of CLEAR network genes.

A limitation of previous studies involving induction of autophagy is their focus on the continual activation of autophagy. While autophagy is generally thought to promote cell survival, as discussed above, under certain conditions sustained autophagic-flux can lead to cell death. Furthermore, in the case of ischemia, prolonged activation of autophagy proteins (e.g., LC3 and BECN1) and vacuoles in response to ischemic stroke/reperfusion in vivo, or oxygen-glucose deprivation (OGD) in vitro leads to significant cell death. Many autophagic processes do not significantly affect cell health until days after the injury, however, indicating that prolonged activation is may be necessary for autophagy-mediated cell death to occur. As another example, constitutive activation of the δ2 glutamate receptor causes Purkinje cell death in Lurcher mice via activation of autophagy. Thus, for elderly tauopathy patients with co-morbid conditions such as ischemia and vascular dementia, sustained activation of autophagy could exacerbate cell death rather than reduce it.

In contrast, the system described herein allows one to maintain spatio-temporal control of autophagy at the transcriptional level. The system is, therefore, tunable and allows one to turn-on/turn-off autophagy in neurons using optical induction based on an engineered light-responsive bacterial transcription factor (Motta-Mena et al. 2014 Nat Chem Biol 10(3):196-202) to drive TFEB expression in a number of different mammalian expression systems that display pathological tau.

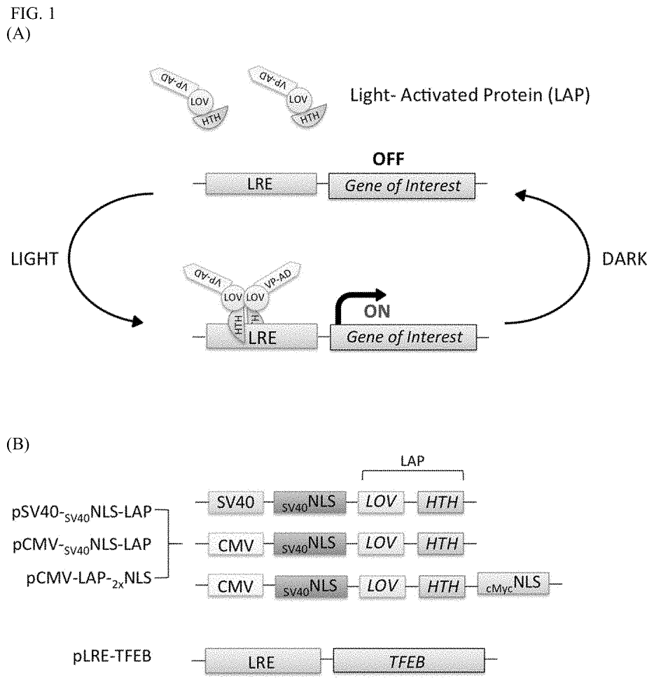

FIG. 1A provides a schematic illustration of the system. A light-responsive element (LRE) controls expression of a gene of interest. The light-activated protein (LAP) binds to the LRE in the presence of light, inducing expression of the gene of interest. In the model exemplary embodiment, a light-activated protein that includes cytomegalovirus (CMV) promoter and the nuclear localization signal (NLS) sequence derived from cMyc shows robust gene expression with blue light. The system also includes an engineered version of EL222, a bacterial transcription factor that contains a Light-Oxygen-Voltage (LOV) protein, which binds DNA when illuminated with blue light (465 nm) (Motta-Mena et al. 2014 Nat Chem Biol 10(3):196-202). This system also contains a dimerizable Helix-Turn-Helix (HTH) DNA-binding domain. In the dark, the LOV domain binds the HTH domain, thus preventing HTH 4a helix dimerization and DNA binding. Upon light stimulation, the HTH domains can now dimerize and bind to a particular DNA sequence that precedes a TATA box in the promoter region. These conversions are spontaneously reversible in the dark, and thus inactivating EL222 dimerization (Motta-Mena et al. 2014 Nat Chem Biol 10(3):196-202; reiterated in FIG. 1A). This system was previously optimized with five copies of the bacterial EL222-binding Clone 1-20 base pairs (C120)₅ sequence (Rivera-Cancel et al., 2012, Biochemistry 51:10024-10034; Zoltowski et al., 2013, Biochemistry 52:6653-6661; Motta-Mena et al. 2014 Nat Chem Biol 10(3):196-202). This consensus site acts like a promoter region for the EL222 binding and drives the expression of any genes inserted downstream of C120 (FIGS. 1A and 1B).

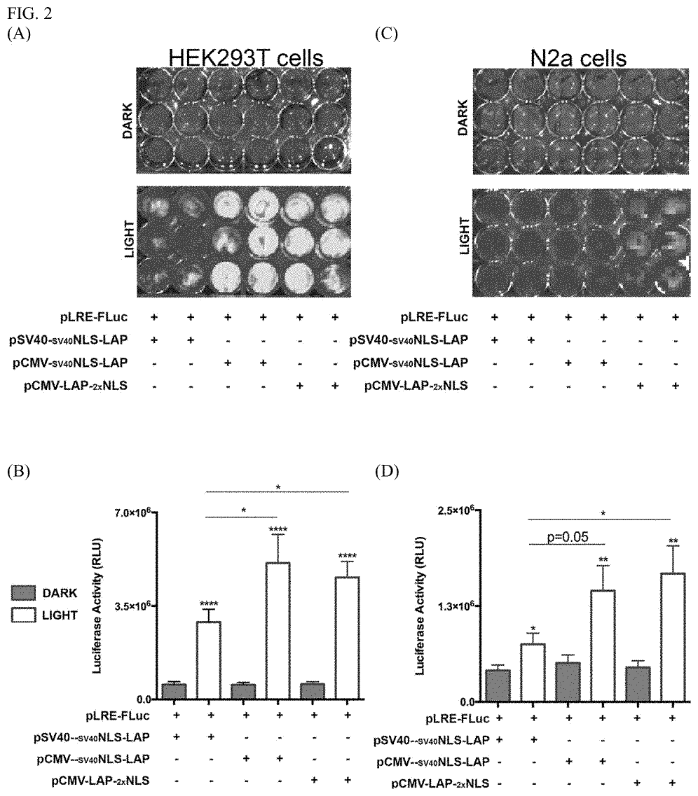

In order to recapitulate the EL222 system, the original two-plasmid system was tested. The system includes plasmid pVP-EL222, (that we call ‘light activated protein’ or ‘LAP’) and pC120-Fluc (Firefly Luciferase reporter that we call ‘light-response element’ or ‘LRE’), where the luciferase gene was inserted downstream of LRE. Robust luciferase activation was observed upon blue light illumination in HEK293T cells (FIGS. 2A and 2B). However, using the original pSV40-_{SV40}NLS-LAP in a neuro2a (N2a) cell line, luciferase expression was less than one-half of that observed in HEK293T cells. Therefore, two different EL222 light-responsive systems were created by replacing the SV40 promoter with a CMV promoter and including an additional cMyc nuclear localization signal (NLS) sequence (FIG. 1B). The modified system produced a two-fold to four-fold increase in luciferase expression upon blue-light stimulation compared to dark controls in both HEK293T and N2a's (FIGS. 2B and 2D). A different degree of luciferase expression also was observed with different promoter and NLS combinations with the pCMV-LAP-2×NLS, showing the robust induction of luciferase expression in N2a cells.

Constitutively active TFEB (i.e., not light inducible) clears various types of pathological tau with equal efficiency in cellular models of tauopathy. Tau gene (MAPT) in humans encodes six different isoforms that are contrasted by exons 2, 3, and 10. Exon 10 encodes a second microtubule binding repeat, thereby resulting in tau with either three (3R—without second repeat) or four (4R—with second repeat) microtubule binding repeats of 31-32 amino acids in the carboxy terminal half. Tau isoforms are also generated in either having one (1N), two (2N), or zero (0N) amino terminal inserts of 29 amino acids in the N-terminal half of the protein. In normal adult brain, the relative amounts of 3R tau and 4R tau are approximately equal, however in neurodegenerative tauopathies, the ratio of 3R:4R is often altered. Besides altered isoform ratios, post-translational modifications, such as phosphorylation in tau, can also affect tau function and contribute to disease pathogenesis in tauopathies.

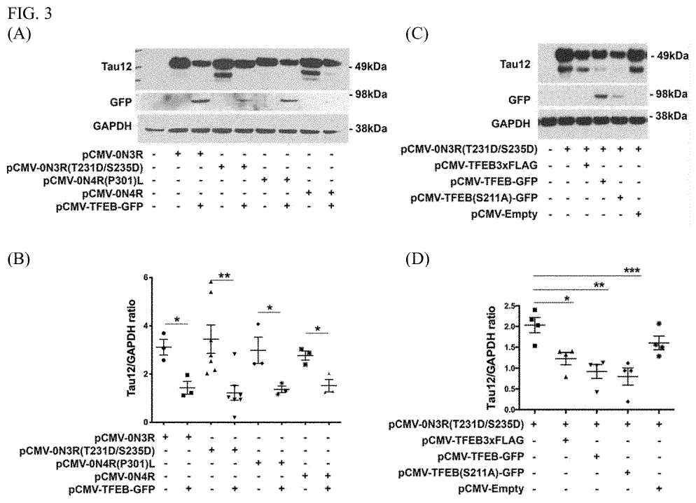

TFEB degrades pTau via beclin-1-dependent autophagy pathway. However, the effectiveness of TFEB on different isoforms of tau with different disease-modifications has not been tested. To test this, TFEB was co-transfected into cells with different forms of tau: either pCMV-0N3R (non-mutant tau, but over-expression can lead to Pick's Disease (PiD)); pCMV-0N3R(T231D/S235D), which mimics hyperphosphorylation on T231/S335 sites and known to disrupt tau's interaction with tubulins; pCMV-0N4R P301L, which causes FTDP-17T; or pCMV-0N4R (non-mutant tau, but over-expression can lead to progressive supranuclear palsy (PSP)). Co-transfection of TFEB with different types of tau lead to consistent reduction in all types of overexpressed total tau levels in N2a cells (FIGS. 3A and 3B), with T231D/S235D phosphorylation-mimicking tau showing the most significant reduction (FIGS. 3A and 3B). Together, these results suggest that TFEB can clear different types of pTau with robust consistency in neuronal cells. Furthermore, since T231 residue can acquire potent neurotoxic conformation called cis-pTau (or ‘Cistauosis’, as a result of phosphorylation of tau at T231), TFEB's role in significantly reducing T231D/S235D species of pTau suggest the potential therapeutic potential of targeting TFEB against tauopathies.

Next, co-expression of T231D/S235D tau with either pCMV-TFEB3×FLAG or pCMV-TFEB-GFP showed that GFP-tagged TFEB has better efficiency in inducing p-Tau reduction than 3×FLAG tagged TFEB (FIGS. 3C and 3D). Given that TFEB has to be nuclear for it to be functionally efficient and to drive the expression of genes in the CLEAR network, the effects of S211 phosphorylation in TFEB in clearing mutant tau were assessed. TFEB-GFP with the S211A mutation (serine 211 changed to alanine), which prevents phosphorylation by mTORC1 and thus promotes TFEB's nuclear entry, had the greatest effect in reducing T231D/S235D mutant pTau (FIGS. 3C and 3D). Together, these results suggest that genetically facilitating the nuclear entry of TFEB enhances the autophagic clearance of T231D/S235D tau.

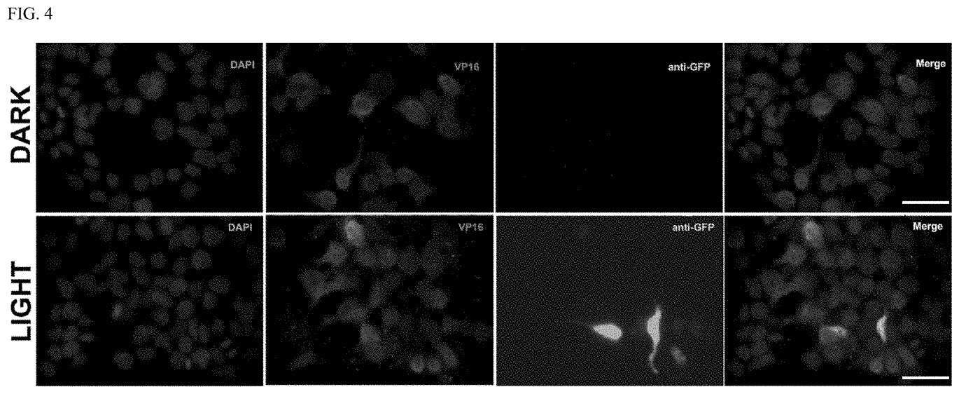

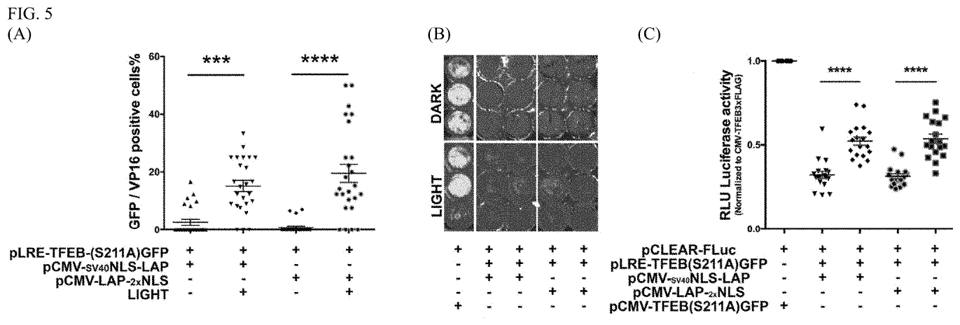

Ontogenetically-expressed TFEB activates CLEAR network genes in neuronal cells. N2a cells were co-transfected with the two different LAPs and pLRE-TFEB(S211A)GFP plasmids. Transfected cells were stimulated with blue light for 12 hours, fixed in 4% PFA, immunostained for VP16 and GFP, and subjected to confocal microscopy double immunofluorescence analysis (for VP16 and GFP). With the substitution of a CMV promoter and additional cMyc NLS, a significant increase of TFEB expression, as revealed by anti-GFP staining, was observed with blue light stimulation compared to dark control (FIGS. 4 and 5A). The VP16 staining was detectable in pCMV-LAP (FIG. 4).

Based on the TFEB's preferential binding to the CLEAR consensus site (5′-GTCACGTGAC-3′; SEQ ID NO:1), a previously published reporter plasmid pCLEAR-FLuc (Cortes et al., 2014, Nat Neurosci September; 17(9):1180-1189) was used to assess the functional activation of Opto-TFEB. The pCLEAR-FLuc plasmid consists of four replicates of the CLEAR consensus sequence upstream of the luciferase gene, serving as a DNA-binding activity readout for TFEB. N2a cells were transiently co-transfected with pCLEAR-FLuc, pLAPs, and pLRE-TFEB(S211A)GFP and then stimulated with blue light overnight (12 hours). Shortly after luciferin treatment, whole cell culture plates were imaged using IVIS Lumina Series III. A significant increase in TFEB DNA-binding activity (enhanced CLEAR-luciferase signal) observed only in cells that optogenetically expressed TFEB with light, and not in the dark control (FIGS. 5B and 5C). Together, the results suggest that Opto-TFEB is functionally active in driving expected transcriptional activity of genes in the CLEAR network.

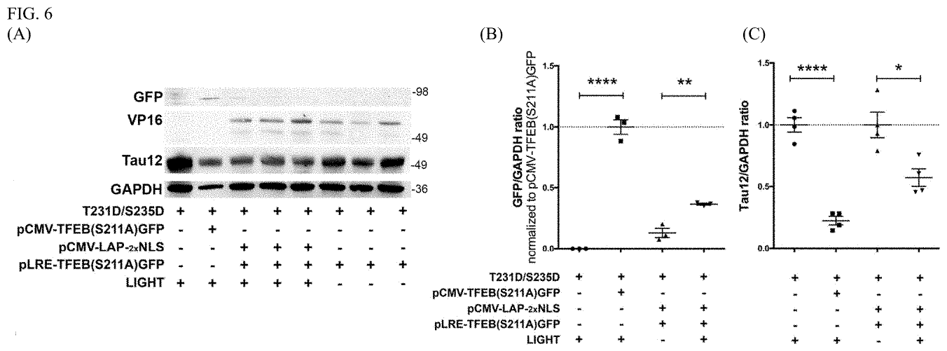

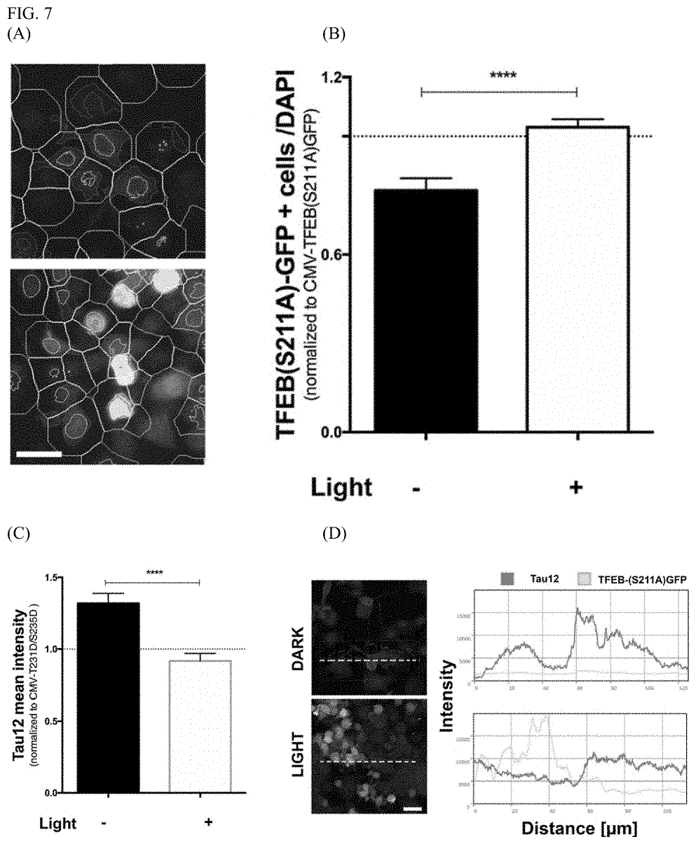

Optogenetically-driven TFEB reduces pathological tau in neuronal cells. Multiple approaches were taken to establish that the Opto-TFEB system can clear specifically phosphorylated species of Tau. First, overexpressed human tau carrying the 0N3R-T231D/S2345D double mutation along with pCMV-LAP2×NLS and pLRE-TFEB(S211A)-GFP in N2a cells. Analysis of TFEB(S211A)-GFP and Tau12 through western blot revealed statistically significant increase in TFEB expression (FIGS. 6A and 6B) and reduction in the levels of total tau (Tau12) (FIG. 6A-C) in light-exposed cells. Confirmatory, unbiased quantitative morphometry analysis for Tau12 levels using high-content, automated CELLOMICS microscopy (Thermo Fisher Scientific, Inc., Waltham, Mass.), revealed a significant decrease in the overall Tau12 intensity in light-exposed Opto-TFEB+ cells compared to Dark controls (FIG. 7A-C). Confocal analysis further confirmed that the fluorescence signals for Tau12 and GFP (from TFEB(S211A)-GFP+ cells) were mutually exclusive and non-overlapping (FIG. 7D). Together, these results demonstrate that light-induced expression of TFEB is capable of reducing overexpressed phospho-mimicking (T231D/S235D) tau levels in neurons.

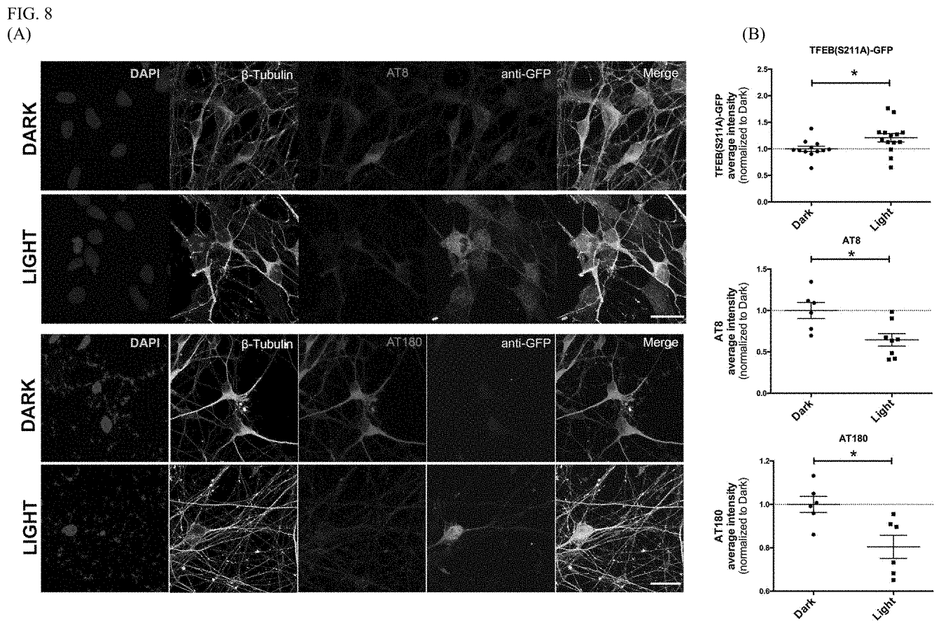

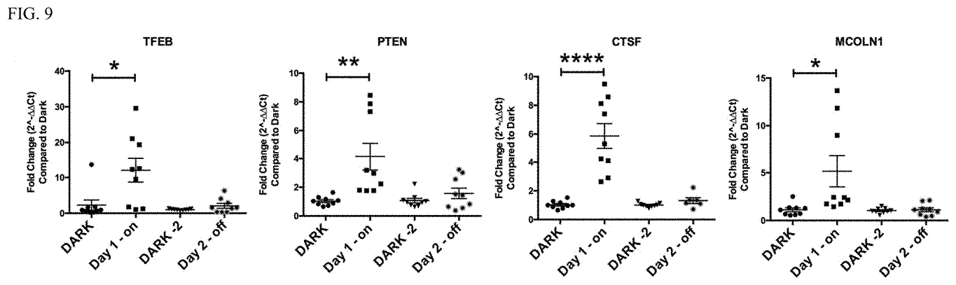

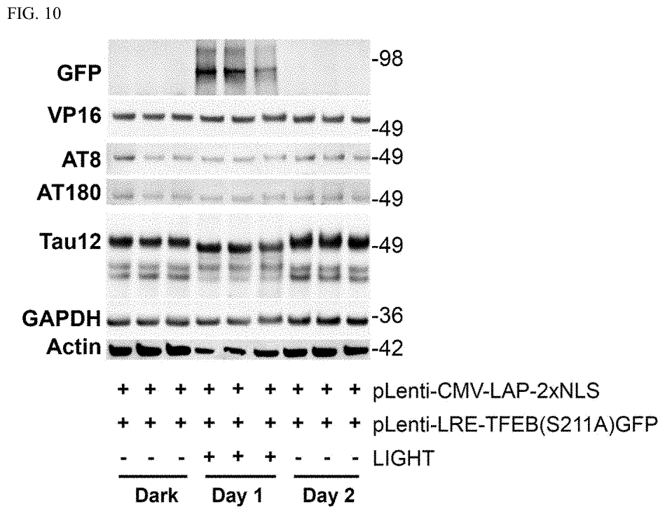

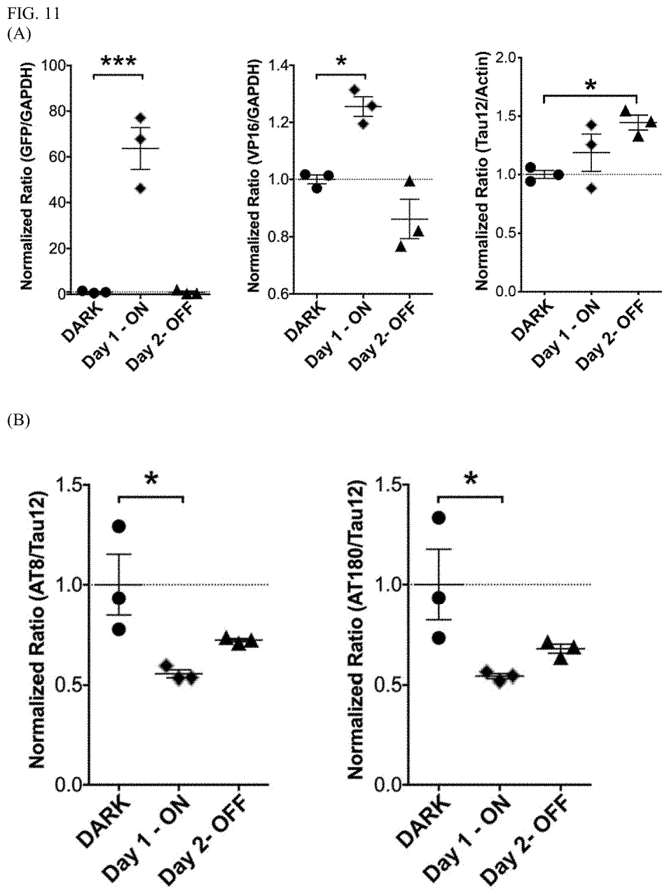

The efficacy of Opto-TFEB was next tested in a previously characterized AD relevant iPSCs cell line called ‘sAD2.1’ (Israel et al., 2012, Nature 482(7384):216-220). The sAD2.1 iPSC neurons (iPSNs) display robust pTau (positive for AT8, AT180, and PHF1) levels. To assess the efficacy of this system in human-relevant model system, Opto-TFEB was tested in induced pluripotent stem cells (iPSC) line from a patient with sporadic AD (sAD2.1). As previously described, the iPSC-derived neurons (iPSNs-sAD2.1 line) displayed robust hyperphosphorylation on Ser202 and Thr231 sites (positive for AT8 and AT180; FIGS. 8A and 8B). To assess the efficacy of Opto-TFEB in sAD2.1 cells, lentiviral Opto-TFEB constructs (pGF1-CMV-LAP2×NLS and pGF1-LRE-TFEB(S211A)-GFP) were created and co-transduced sAD2.1 iPSNs. Similar to results in N2a cells, light-exposed iPSNs displayed a significant increase in TFEB-GFP and a consequential decrease in both AT8 and AT180 p-Tau levels compared to Dark controls (FIGS. 8A and 8B). Lastly, to assess the temporal dynamics of Opto-TFEB, the light-dark activityof Opto-FEB was analyzed across two days. On day one, iPSNs were stimulated with light overnight and an identical plate of iPSNs was left in the dark. After 12 hours of light stimulation, a row of cells was collected for analysis. The following day, the light was left off and another row of cells were collected for analysis 24 hours after the first collection. First, mRNA levels of three known TFEB targets, PTEN, CTSF, and MCOLN1, were measured (FIG. 9). On day one, there was a significant increase in TFEB expression with light and up-regulation of TFEB target genes compared to Dark (FIG. 9). The mRNA levels of TFEB-target genes reduced back to basal levels after a day of no light. Western blot analysis to detect total protein levels revealed p-Tau (AT8 and AT180) was significantly reduced (FIG. 11B). Notably, while the total tau levels were unaltered, Tau12+ bands showed slightly faster migration (FIG. 10). On day two, levels of TFEB(S211A)-GFP and TFEB targets were down to dark levels, however the AT8+ and AT180+p-Tau levels seem to have gradually raised but still remained significantly lower than their starting levels (FIG. 11A). Taken together, for the first time, these results suggest that light-induced, optogenetic-based expression of TFEB can reduce p-Tau in a human relevant iPSN tauopathy model.

TFEB is a master transcriptional regulator of autophagy and lysosome biogenesis. However, studies have revealed TFEB constitutes and interacts with a variety of biological functions, including the inflammatory process, stress responsive pathways, oxidative stress, and metabolic regulation. Therefore, a system that employs TFEB as a therapeutic target cannot involve constitutive expression of TFEB or maintain TFEB in an active, nuclear state.

The system described herein uses a light-inducible gene expression system that offers spatial and temporal control over expression of the gene controlled by the system. In a model embodiment, TFEB expression was controlled in human-relevant mouse tauopathy models and in human AD iPSCs derived into mature neurons. The system described herein, therefore, provides a transient ‘on/off’ activation/deactivation mechanism using a novel blue-light-inducible TFEB gene expression system.

This disclosure also describes effective enhancement of the autophagy flux via mutation of an mTORC1 site, S211A. The mutation facilitates nuclear entry of TFEB and robust clearance of p-Tau in the human AD derived iPSNs. AT8 and AT180 levels increased on Day 2 (when the Light is off), accompanied by notable tau buildup due to Dark (no induction of autophagy beyond basal level). The production of p-Tau on the day after the light was turned off establishes that the Opto-TFEB system provides a spatio-temporal control of gene expression. When autophagy is turned off, the kinases are re-activated and/or re-accumulation of hyperphosphorylated tau occurs.

While autophagy is generally thought to promote survival as discussed above, certain conditions can lead to autophagic-mediated cell death. For example, constitutive activation of the δ2 glutamate receptor is thought to cause Purkinje cell death by activating autophagy processing. Moreover, prolonged activation of autophagy proteins (e.g., LC3 and BECN1) and vacuoles can occur in response to ischemic stroke/reperfusion in vivo and/or or oxygen-glucose deprivation (OGD) in vitro. Many autophagic processes do not significantly affect cell health until days after the injury, indicating that prolonged activation mediates cell death. Furthermore, administering the autophagy-inhibiting chemical 3-MA significantly reduces cell death in cells that exposed to OGD or ischemic injury. Lastly, administration of Wortmannin reduced autophagic processing and improved memory in animals with vascular dementia. Thus, for elderly tauopathy patients who may be at enhanced risk for other types of brain damage such as ischemia and vascular dementia, chronic induction of autophagy could exacerbate cell death rather than reduce it. Thus, in some cases, the light dose may need to be carefully titrated.

Thus, this disclosure describes the construction, expression, and functional efficacy of neuronal Opto-TFEB in inducing the expression of CLEAR network genes for the induction of autophagy-lysosomal pathways and p-Tau clearance. The tunable Opto-TFEB expression system also may work in other cell types within the CNS. While described herein in the context of an exemplary embodiment in which the Opto-TFEB system specifically targets one particular isoform of tau with phosphorylation-mimicking mutations (0N3R-T231D/S235D), the tau isoform directly relevant to AD, the systems and methods described herein can involve the targeting of other isoforms of tau such as, for example, 1N3R, 1N4R, etc.

In another aspect, this disclosure describes using an optogenetic gene expression system in vivo. The genetic construct can be delivered to target cells using any conventional gene therapy delivery method including, but not limited to, nanoparticle-based methods and/or viral vectors designed to include a neuron-specific promoter.

The gene therapy delivery may be by any suitable conventional route of administration such as, for example, a systemic and/or an intranasal approach. Alternative delivery strategies include inserting a probe, similar to probes used for deep-brain stimulation (DBS) for Parkinson's disease therapeutics, and then introducing the optogenetic gene expression system construct directly into the brain. Yet another approach can involve the use of a lentiviral vector previously described (Palfi et al., Lancet. 2014 Mar. 29; 383(9923):1138-46. doi: 10.1016/S0140-6736(13)61939-X. Epub 2014 Jan. 10.).

Once the optogenetic gene expression system is introduced into cells of a subject, the subject can be subjected to light as previously described (Binder et al., PLoS One. 2020; 15(3): e0230026. doi: 10.1371/journal.pone.0230026). The light may be delivered invasively (e.g., via optical fiber) or noninvasively. Noninvasive systems may involve an infrared-based system in which the optogenetic gene expression construct is designed to be responsive to infrared light. For therapy targeting neuronal tissues, an noninvasive light source may be integrated into a headpiece (e.g., a hat, helmet, or other device worn or placed on or about the head).

The duration of the exposure to light that activates the optogenetic gene expression system can vary depending on the stage of disease, extent of neuronal damage, and strength of gene expression induced by exposure to light. In some cases, for example, the subject may be exposed to light for about an hour, one or two times a month initially. Frequency and/or duration of treatment can be increased or decreased according to the subject's response to the initial treatment.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Vector Construction

All constructs (Table 1) were cloned using a HIFI Assembly Kit (New England Biolabs, Inc., Ipswich, Mass.) with restriction enzymes and PCR amplification. Briefly, the original episomal plasmids (pVP-EL222 and pGL4-C120-mCherry; Motta-Mena et al., Nat Chem Biol. 2014; 10(3):196-202. 10.1038/nchembio.1430) were cloned into different backbones with subsequent promoters and/or gene of interest; pN1-CMV-TFEB-GFP (Addgene, Inc., Watertown, Mass.). Newly cloned episomal plasmids were then additional cloned into lentivector backbone, pGF1-Nf-KB-EF1-Puro (System Biosciences, Inc., Palo Alto, Calif.). A site-directed mutagenesis kit Q5, New England Biolabs, Inc., Ipswich, Mass.) was used to make mutations (S142A and S211A) in TFEB gene. All Tau constructs used: 1) pRC/CMV-0N3R-tau (human tau with three microtubule-binding repeats with no N-terminal inserts); 2) 0N4R-tau (human tau with four microtubule-binding repeats with no N-terminal inserts); 3) 0N4R-P301L (human tau with four microtubule-binding repeats with P301L FTDP-17T mutation); 4) 0N3R-T231D/S235D.

TABLE 1
Light-responsive plasmid constructs

| | | References/ |
|---|---|---|
| Name | Description | Source |
| pGL4-SV40- | Bacteria | 1 |
| VP-EL222 | Transcription | |
| | Factor, EL222, | |
| | LOV domain. | |
| pC120-MCH | mCherry reporter | 1 |
| pC120-FLuc | Firefly Luciferase reporter | 1 |
| pN1-CMV- | Constitutive TFEB- | Addgene |
| TFEB-GFP | GFP reporter | #38119 |
| pN1-CMV- | Constitutive TFEB with | |
| TFEB(S211A)-GFP | (S211A) mutation- | |
| | GFP reporter | |
| pN1-LRE- | LRE-Flag reporter | Light response |
| TFEB3xFLAG WT | | element |
| pN1-LRE- | LRE-Flag reporter | |
| TFEB(S142A)3xFLAG | | |
| pN1-LRE- | LRE-GFP reporter | (generated for |
| TFEB-GFP WT | | the present study) |
| pN1-LRE- | LRE-GFP reporter | |
| TFEB(S211A)-GFP | | |
| pGF1-LRE- | Lenti-LRE-TFEB- | |
| TFEB(S211A)-GFP | GFP reporter | |
| pN1-CMV-EL222 | LAP, CMV promoter, | Light-activated |
| | Sv40 NLS N term | protein |
| pN1-CMV- | LAP, CMV promoter, | |
| EL222-₂ₓNLS | Sv40-NLS, | |
| | and cMyc NLS | |
| pGF1-CMV- | Lenti-LAP | |
| EL222-₂ₓNLS | | |

1 Motta-Mena et al., Nature Chem Biol. 2014; 10(3): 196-202. doi: 10.1038/nchembio.1430. PubMed PMID: 24413462; PubMed Central PMCID: PMC3944926

### Cell Lines

HEK293T (ATCC #CRL-3216) and Neuro-2a (ATCC #CCL-131) cells were maintained at 37° C. in 5% CO2 in DMEM supplemented with 10% FBS, 5% penicillin/streptomycin, and grown in 24-well plates. For transient transfections, cells were split the day before—1-4×10⁵ cells/well, therefore 70-80% confluence the following day. Before transfection, media was replaced with phenol red free media, (FLUOROBRITE DMEM; Thermo Fisher Scientific, Inc., Waltham, Mass.). Cells were then transfected with LIPOFECTAMINE 2000 (Invitrogen, Carlsbad, Calif.) by manufacturer's protocol. Dilutions of various plasmid concentrations were as followed for a 24-well plate: pLAPs—2000 ng/μL, pLREs—500 ng/μL, pCMV-TFEBs—500 ng/μL, pCMV-hTaus—1000 ng/μL, pCLEAR-FLuc—500 ng/μL, maintaining a 1:4 ratio of LRE to LAP.

### Induced Pluripotent Stem Cells

sAD2.1 (Israel et al., Nature 2012; 482(7384):216-20. doi: 10.1038/nature10821. PubMed PMID: 22278060; PubMed Central PMCID: PMC3338985; Coriell Institute for Medical Research, Camden, N.J.), iPSCs from Fibroblast NIGMS Human Genetic Cell Repository Description: ALZHEIMER DISEASE; AD Affected: Yes. Gender: Male. Age: 83 YR (At Sampling). Race: Caucasian. Control line: Axolbio #ax0018 (iPSC-Derived Neural Stem Cells; Male; Axol Bioscience Ltd., Cambridge, UK)

Briefly, iPSCs were maintained in mTESR+supplement (StemCell Technologies, Inc., Vancouver, CA). A neuron differentiation kit (StemCell Technologies, Inc., Vancouver, CA) to differentiate neurons. Later, medium was changed to BRAINPHYS without phenol red was used for optical induction. Neural progenitor cells seeded at 1.5×10⁴ cells/cm² for maturation.

### Light Induction

Twelve hours post transfection, an in-house blue LED device (465 nm, strip of LEDs glued to PCB board) was placed 8 cm or 16 cm above the plate. The intensity of the light received by cells was measured to be to 8 W/m² and verified, using the LI-190 Quantum Sensor and LI-250A light meter (LI-COR Biosciences, Lincoln, Nebr.). The LED strips were connected to a remote controller for varying on/off patterns to best match a cycle of 20 seconds on and 60 seconds off. The control plate was kept in a PCB blackout box with breathable air slots, (a shelf in the incubator, above and away from the light source shelf). For transiently transfected cells, 24 hours post-transfection, samples were collected/fixed for analysis.

### Lentivirus Production and Luciferase Assay

Using HEK293 Ts seeded in 100 mm plates, lentiviral transgenes were cloned into the pGF1-EF1-Puro backbone. Lentiviral packaging vectors pMD.2 and pPAX2 (Invitrogen, Carlsbad, Calif.) were used. Cells were transfected with plasmid mix using a CaPO4 precipitation method as previously described (Tiscornia et al. 2006 Nat Protocols 1(1):241-245). After a 48-hour interval, the viral supernatant was then filtered through 0.45 μm membranes and mixed overnight with a LENTI-X concentrator (Takara Bio USA, Inc., Mountain View, Calif.). The next day, samples were centrifuged at 1,500×g for 45 minutes at 4° C. An off-white pellet is then resuspended in subsequent media, ex: if iPSNs, then neurobasal. Lentiviral titer was measured using cat #631280 LENTI-X GOSTIX Plus (Takara Bio USA, Inc., Mountain View, Calif.).

Lentiviral Transduction on iPSNs: an IFU of 1×10⁶/mL were added to the neurons to make ˜MOI=2. sAD2.1 neural progenitor cells were transduced 24 hours after plating on poly-ornithine/laminin coated coverslips following StemCell maturation protocol (StemCell Technologies, Inc., Vancouver, CA). Subsequently, two weeks after transduction, (Day 40) iPSNs were subjected to light stimulation (12 hours) or kept in the dark. Samples were then collected/fixed for analysis.

For Firefly luciferase activities, 4XCLEAR-luciferase reporter plasmid (Addgene, Watertown, Mass.) was used. D-luciferin, potassium salt (Thermo Fisher Scientific, Inc., Waltham, Mass.) was reconstituted in water and was added (1:100) to each well, 3-4 minutes after adding the substrate, 24-well plate samples were analyzed through the IVIS Lumina Series II with system software.

### Western Blotting (WB) and Immunocytochemistry (ICC)

Cells were lysed by RIPA buffer (Thermo Fisher Scientific, Inc., Waltham, Mass.), incubated on ice for 30 minutes then centrifuged at 20,000×g for 15 minutes. Cell lysate supernatants were then sonicated for 20 seconds at 30%, then subjected to SDS-PAGE, transferred to PVDF membranes, and detected using the ECL method (Pierce, Thermo Fisher Scientific, Waltham, Mass.). Protein levels were quantified using ImageJ (National Institute of Health). Antibodies included: tau12, GAPDH, FLAG, GFP, TFEB, ATB, AT180, LC3B, LAMP 1.

For immunocytochemistry (ICC) studies, cells were plated on coverslips coated with laminin. Once cells were ready for fixation, they were fixed in 4% PFA and blocked with 0.2% triton and 10% donkey serum. The coverslips were incubated in primary antibody overnight in 4° C. (5% DS), washed, then secondary antibodies were incubated for one hour at room temperature. After unbound secondary antibodies were washed, the coverslips were incubated in DAPI for 10 minutes, and mounted to slides using FLUOROMOUNT (Thermo Fisher Scientific, Inc., Waltham, Mass.). Immunofluorescence confocal microscopy was carried out using Zeiss LSM 510 Meta microscope. Histology and profile analysis was performed using ZEISS ZEN imaging Software.

### Gene Expression Analysis

RNA from cells was extracted using the TriZOL reagent as described by the manufacturer (Thermo Fisher Scientific Inc., Waltham, Mass.). Total RNA (20 ng/μL) was converted to cDNA using the High Capacity cDNA Reverse Transcription kit (Thermo Fisher Scientific Inc., Waltham, Mass.) and amplified using specific TaqMan assays (Thermo Fisher Scientific Inc., Waltham, Mass.). GAPDH (Thermo Fisher Scientific Inc., Waltham, Mass.) was used as a housekeeping gene for normalization. qRT-PCR assays were run on the STEPONEPLUS real-time PCR System (Thermo Fisher Scientific Inc., Waltham, Mass.) and the statistical analyses were performed using PRISM software (GraphPad Software Inc., San Diego, Calif.).

### CELLOMICS-Based High-Content Imaging Analysis

Cells were plated in 96-well plates transiently transfected with pCMV-T231D/S235D (phosphorylation-mimicking tau), pCMV-LAP2×NLS, and pLRE-TFEB(S211A)-GFP. Twenty-four hours later, cells were incubated with conditioned medium from BV-2 cells, then subsequently induced with light (470 nm) for 12 hours. Cells were fixed in 4% PFA and blocked with 0.2% triton and 10% donkey serum. The cells were incubated with primary antibody for one hour at room temperature (5% DS), washed, then incubated with secondary antibody for one hour at room temperature. After washing unbound secondary antibody, the cells were incubated in DAPI for 10 minutes and analyzed using a CELLOMICS instrument (Thermo Fisher Scientific, Inc., Waltham, Mass.).

### Statistics

Unless otherwise indicated, comparisons between the two groups were done via unpaired t-test; comparisons between multiple treatment groups were done via one-way or two-way analysis of variance (ANOVA) with indicated multiple comparisons post-hoc tests. All statistical analyses were performed using PRISM software (GraphPad Software Inc., San Diego, Calif.).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A genetic expression system comprising:
a polynucleotide that encodes Transcription Factor EB (TFEB) under transcriptional control of a promoter; and
a polynucleotide that encodes a light-activatable protein that binds to the promoter in the presence of light but does not bind to the promoter in the absence of light.

2. The genetic expression system of claim 1, wherein the promoter is the cytomegalovirus (CMV) promoter.

3. The genetic expression system of claim 1, wherein the TFEB includes, at its C-terminus, the cMyc nuclear localization signal (NLS)

4. The genetic expression system of claim 1, wherein the light-activatable protein comprises a complex that includes:
Light-Oxygen-Voltage (LOV) protein; and
a dimerizable Helix-Turn-Helix (HTH) DNA-binding domain.

5. A cell comprising the genetic expression system of claim 1.

6. The cell of claim 5 wherein the cell is a neuron.

7. The cell of claim 6 wherein the neuron is a cell at risk of displaying a tauopathy.

8. A method of treating a neuron at risk of displaying a tauopathy, the method comprising:
introducing into the cell the genetic expression system of claim 1;
exposing the cell to light effective to cause the light-activatable protein to bind to the promoter, thereby expressing TFEB.

9. The method of claim 8, wherein exposing the cell to light comprises exposing the cell to light for a period sufficient for the genetic expression system to produce a protein that promotes autophagy of the neuron.

10. The method of claim 9, wherein promoting autophagy of the neuron comprises reducing pathological tau protein in the neuron.
